(19) United States
(12) Patent Application Publication
(10) Pub. No.: US 2006/0247788 A1
(43) Pub. Date: Nov. 2, 2006

(54) Total ankle arthroplasty

(75) Inventor: Steven D. K. Ross, Orange, CA (US)

Correspondence Address: CROWELL & MORING LLP;INTELLECTUAL PROPERTY GROUP, P.O. BOX 14300, WASHINGTON, DC, 20044-4300, US

(73) Assignee: The Regents of the University of California, Oakland, CA (US)

(21) Appl. No.: 11/393,048

(22) Filed: Mar. 29, 2006

Related U.S. Application Data
(60) Provisional application No. 60/667,501, filed on Mar. 31, 2005.

(51) Int. Cl. A61F 2/42

(52) U.S. Cl. 623/21.18

(57) ABSTRACT

A three-component system is used for total ankle arthroplasty in which a tibial prosthesis component is fixed to a distal tibia end, a talar prosthesis component is fixed to a talus, and a bearing component is positioned between and in contact with the tibial prosthesis component and the talar prosthesis component. In one embodiment, the natural curvature of the distal tibia end is maintained rather than creating a flat surface to which to adhere a prosthetic component. In still another embodiment, a lateral or medial incision is used to insert the prosthetic components of the total ankle arthroplasty.

### CROSS-REFERENCE TO RELATED APPLICATION

This application is related to and claims priority benefit of U.S. Provisional Patent Application Ser. No. 60/667,501 filed on Mar. 31, 2005, which is hereby fully incorporated by reference.

### BACKGROUND

1. Field of the Invention

The present invention relates to ankle replacements in general, and more particularly to a total ankle replacement system which is inserted through a medial or lateral incision as opposed to the traditional anterior incision.

2. Background

Over the years there have been several efforts to provide a total ankle replacement solution. However, all such current solutions have significant drawbacks, including complications relating to aseptic loosening, delayed wound healing, wound dehiscence, and prosthetic subsidence. Other problems include prosthesis migration and loosening, and osteolysis at the tibial component.

One comment element of all the current ankle replacement solutions is that they are installed through an anterior incision in the ankle. The consequence of this is that the tibia must be cut flat to allow anterior insertion of the tibial component. This disrupts the boney architecture in the distal plafond and places the component into softer less stable bone. This leads to easier subsidence of the component and potential failure. The anterior incision also has a higher incidence of wound breakdown in the perioperative period.

There is, therefore, a need in the field for a total ankle replacement system that reduces the occurrence of subsidence while improving the wound healing rate.

### BRIEF SUMMARY OF THE INVENTION

Disclosed and claimed herein are systems, implants and kits for total ankle arthroplasty. In one embodiment, a system includes a tibial prosthesis component configured to be fixed to a distal tibia end having approximately a natural radius of curvature, and a talar prosthesis component configured to be fixed to a talus. The system further includes a bearing component configured to be positioned between and in contact with the tibial prosthesis component and the talar prosthesis component.

Other aspects, features, and techniques of the invention will be apparent to one skilled in the relevant art in view of the following detailed description of the invention.

### BRIEF DESCRIPTION OF THE DRAWINGS

FIG. 1 is a fragmentary front elevational view of a foot and traditional incision location;

FIG. 2 is a side view a total ankle arthroplasty in accordance with one embodiment of the invention;

FIG. 3 is a perspective view of one embodiment of the tibial prosthesis component of FIG. 2;

FIG. 4 is a cross-section view of one embodiment of the tibial prosthesis component of FIG. 3;

FIG. 5 is a side view of one embodiment of the bearing component of FIG. 2 shaped in accordance with the principles of the invention;

FIG. 6 is a perspective view of one embodiment of the talar prosthesis component of FIG. 2;

FIGS. 7A-7B are two embodiments of the talar prosthesis component of FIG. 2 viewed from a bottom perspective; and

FIG. 8 is a perspective view of one embodiment of the bearing component of FIG. 2.

### DETAILED DESCRIPTION OF EXEMPLARY EMBODIMENTS

One aspect of the invention is to provide an implant, system and/or kit for performing a total ankle arthroplasty using a three-component system. In one embodiment, the system or kit includes a tibial prosthesis component fixed to a distal tibia, a talar prosthesis component fixed to a talus, and a bearing component positioned between the tibial prosthesis component and the talar prosthesis component.

Another aspect of the invention is to provide a total ankle arthroplasty system/kit/implant which preserves the natural curvature of the distal tibia rather than creating the typical flat surface to which to adhere a prosthetic component. In one embodiment, an approximately uniform layer of bone material may be removed from the distal tibia prior to insertion of a tibial prosthesis component. This enables the axis of saggital plane rotation of the ankle to remain in approximately the same plane, as opposed to traditional methods which can shift the axis of rotation by flattening the distal tibia. Maintaining the axis of rotation may, in turn, maintain the natural load distribution relationship between the surrounding ligament structure and the ankle bone structure. This would reduce long-term wear-related complications.

In another embodiment, one or more grooves may then be cut into the bone in the laterial/medial direction. These grooves may be used to secure a tibial prosthesis component which is designed with corresponding protrusions that interconnect with the tibial grooves.

Another aspect of the invention is to utilize a lateral or medial incision in which to insert the prosthetic components of the total ankle arthroplasty. In one embodiment, lateral or medial incisions may reduce the incidence of wound complications and improve wound healing time.

Yet another aspect of the invention is to secure a tibial prosthesis component and/or a talar prosthesis component using a system of mounts and screws. In one embodiment, one or more screws may be inserted through mounts attached to one or both of the aforementioned prosthesis components. Such screws may then be inserted into and secured by bone.

Still another aspect of the invention is to provide a groove/protrusion interface between a talar prosthesis component and a bearing component which enables and facilitates a pivot function. In one embodiment, the talar prosthesis component is designed with two protruding members shaped in a curved or semicircular design. A corresponding bearing component is then designed with corresponding grooves to accommodate the protruding members. In another embodiment, the length of the grooves is larger than the length of the corresponding protrusions so as to enable the bearing component to pivot in relation to the talus. In one embodiment, this rotation is able to approximate the normal axis of rotation of the hindfoot.

As used herein, the terms “a” or “an” shall mean one or more than one. The term “plurality” shall mean two or more than two. The term “another” is defined as a second or more. The terms “including” and/or “having” are open ended (e.g., comprising). Reference throughout this document to “one embodiment”, “certain embodiments”, “an embodiment” or similar term means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner on one or more embodiments without limitation.

The term “or” as used herein is to be interpreted as inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

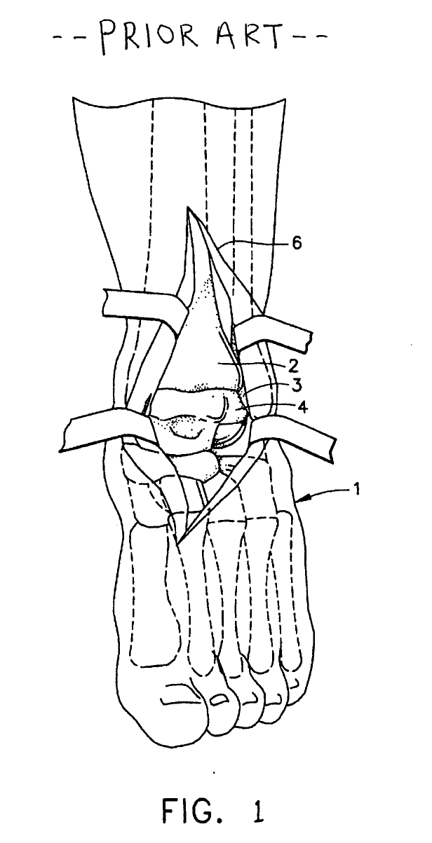

FIG. 1 depicts a foot 1 having a tibia 2, fibula 3 and talus 4. FIG. 1 illustrates how the incision 6 of a typical ankle replacement is made. In particular, the incision is made in the anterior portion of the foot 1 and is often about 10 cm in length or more. In contrast, one aspect of the invention is to use a lateral or medial incision in which to insert a prosthetic ankle system of the invention. This may have the benefit of reducing healing time and minimizing the risk of wound breakdown during the perioperative period.

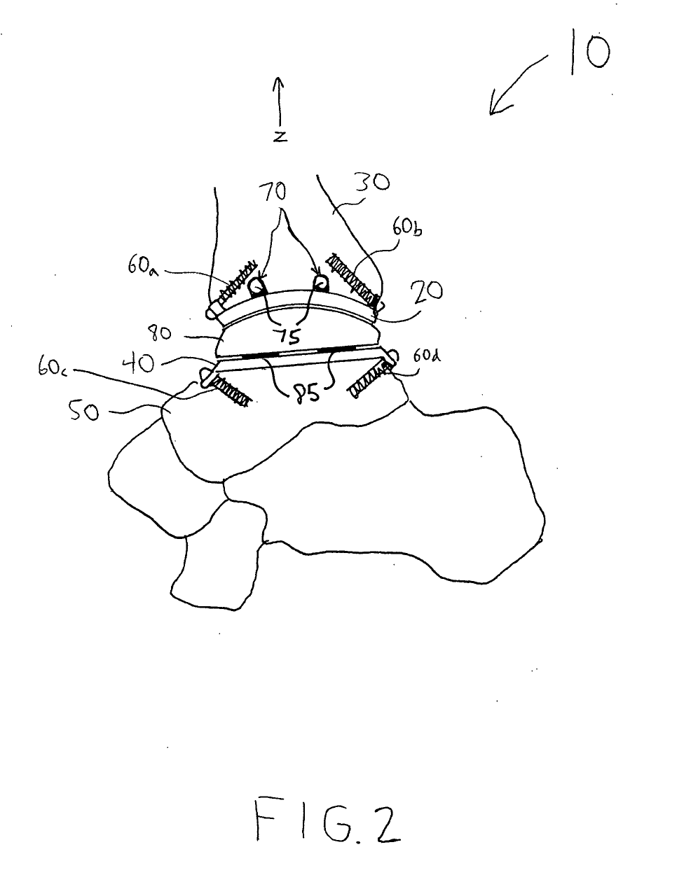

FIG. 2 depicts one embodiment of an ankle replacement system consistent with the principles of the invention. In this embodiment, ankle 10 is shown from a side (i.e., medial or lateral) with a tibial prosthesis component 20 fixed to a distal tibia 30, a talar prosthesis component 40 fixed to a talus 50, and a bearing component 80 positioned between and in contact with the tibial prosthesis component 20 and the talar prosthesis component 40.

As shown, the tibial prosthesis component 20 has a first major tibial surface fixed to a distal tibia end 30, and a second major tibial surface opposite the first major tibial surface. The talar prosthesis component 40, on the other hand, has a first major talar surface fixed to the talus 50, and a second major talar surface opposite this first major tibial surface. Disposed therebetween is the bearing component 80, which is positioned between and in contact with the second major tibial surface and the second major talar surface.

In one embodiment, the tibial prosthesis component 20 and talar prosthesis component 40 are secured to the distal tibia 30 and talus 50, respectively, using a surgically acceptable cement such as methyl methacrylate. Instead of, or in addition to, the tibial prosthesis component 20 and talar prosthesis component 40 may also be secured to the distal tibia 30 and talus 50 using one or more screws 60a-60d, as shown in FIG. 2. It should be appreciated that the number and/or orientation of such screws 60a-60d may vary.

Prior to securing the tibial prosthesis component 20 and talar prosthesis component 40 to the distal tibia 30 and talus 50 respectively, the distal end of tibia 30 and talus 50 may be prepared by cutting away or otherwise removing a relatively small about of bone material. While in one embodiment, approximately 3 mm of bone material may be cut away from the distal tibia 30 and approximately 10 mm from the apex of the original talus 50, it should equally be appreciated that more or less bone material similarly may be removed prior to securing the two prosthesis components 20 and 40. For example, the amount of bone material which may be removed from the distal end of the tibia 30 may range from about 3 mm to about 5 mm, while the amount of bone material which may be removed from the talus 50 may range from about 6 mm to about 10 mm.

Continuing to refer to FIG. 2, it should further be seen that the shape of the cut made to the end of the distal tibia 30 may be made such as to preserve the radial nature of the joint as much as possible, thus maintaining the natural load bearing distribution of the surrounding ligaments and bone components. In other words, unlike with conventional approaches, one aspect of the invention is to avoid altering the natural concave shape of the end of the distal tibia 30.

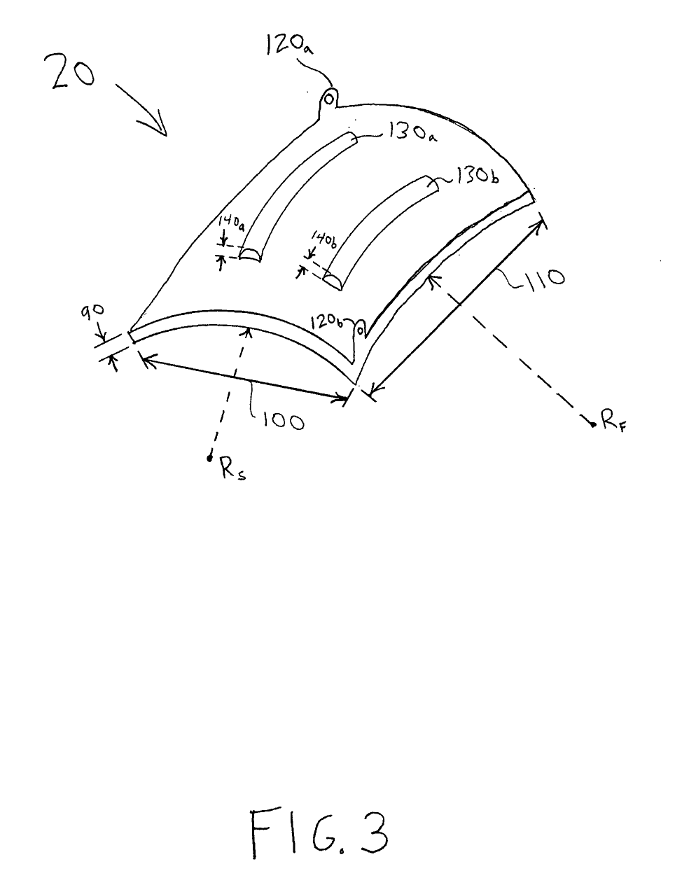

As described above, the tibial prosthesis component 20 and talar prosthesis component 40 may be secured to the distal tibia 30 and talus 50 using any surgically acceptable bone cement and/or screws 60a-60d. However, in addition the tibial prosthesis component 20 may be further secured to the tibia using one or more protrusions 70, which are oriented to interconnect with corresponding grooves 75 in the distal tibia 30. FIG. 3 depicts the protrusions 70 as being snuggly secured in the corresponding grooves 75. In one embodiment, these grooves 75 are cut into the distal tibia 30 from a lateral or medial direction through the same incision used to insert the two prosthesis components 20 and 40. While the protrusions 70 and corresponding grooves 75 are depicted in FIG. 2 as being cylindrical in shape, numerous other configurations would also be consistent with the principles of the invention. In addition, while two protrusion/groove pairs are depicted, it should equally be appreciated that fewer or more protrusion/groove pairs may be used consistently with the principles of the invention.

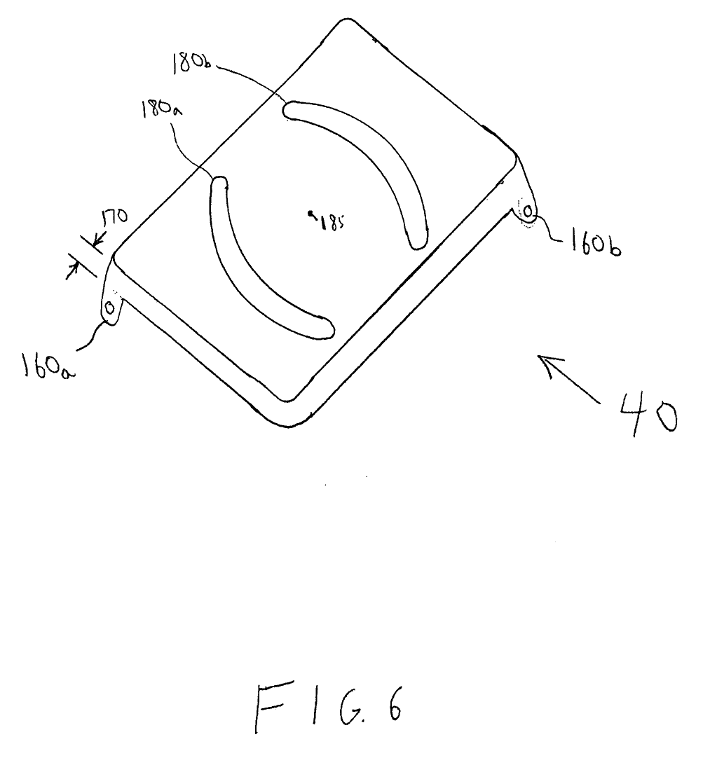

As will be described in more detail below with reference to FIG. 6, the talar prosthesis component 40 and bearing component 80 may also have one or more protrusion/groove interface 85. While the previously-described protrusion/groove interconnection between the tibial prosthesis component 20 and the bearing component 80 is intended to further secure the prosthesis component 20, the protrusion/groove interface 85 between the talar prosthesis component 40 and bearing component 80 serves to provide a pivoting function. That is, a protruding member(s) from the top of the talar prosthesis component 40 having a circular or curved shape may interface with a correspondingly shaped groove in the bottom of the bearing component, thereby providing a pivoting interface about the z-axis, as shown on FIG. 2. It should be appreciated that this radius of curvature may vary significantly and still be consistent with the previously-described pivoting function.

While the tibial prosthesis component 20 and talar prosthesis component 40 may be made of numerous different materials, in some embodiments they may be made of cobalt-chrome, stainless steel, titanium, etc. Similarly, the bearing component 80 may be made of high density polyethylene. Other materials known in the art may similarly be used. In one embodiment, a kit for performing total ankle arthroplasty may contain at least the tibial prosthesis component 20, talar prosthesis component 40 and bearing component 80. Other components may also be included.

Referring now to FIG. 3, depicted is one embodiment of the tibial prosthesis component 20 described in FIG. 2. In this embodiment, the prosthesis component 20 has a thickness 90, which in one embodiment is approximately 3 mm. However, thickness 90 may similarly range from about 3 mm to about 5 mm. In addition, the tibial prosthesis component 20 is shown with a radius of curvature Rₛ along the medial/lateral axis, which in one embodiment approximates the natural curvature of the distal tibia 30. While in one embodiment an exemplary value for Rₛ is 36 mm, it should equally be appreciated that Rₛ may be any value which helps to preserve the natural concave shape of the distal tibia 30. In one embodiment, Rₛ may range from about 30 mm to about 40 mm. The tibial prosthesis component 20 may also have a second radius of curvature R_{F} along the anterior axis to provide improved weight distribution. In one embodiment, R_{F} is 72 mm, although this value may equally be higher or lower. In another embodiment, R_{F} may range from about 50 mm to about 100 mm.

Continuing to refer to FIG. 3, tibial prosthesis component 20 is further shown as having a width 100 along the medial/lateral axis and a width 110 along the anterior axis. While in one embodiment widths 100 and 110 may be approximately equal (e.g., approximately 40 mm), in other embodiments they may be different depending on the dimensions of the particular ankle to be replaced.

Tibial prosthesis component 20 is also shown with optional mounts 120a and 120b. As previously mentioned, the tibial prosthesis component 20 may be further secured to the tibia 30 using screws 60a-60d. To that end, mounts 120a and 120b may be usable to secure the tibial prosthesis component 20 to the distal tibia 30. While in one embodiment, mounts 120a and 120b are approximately 7 mm in height, they may equally be higher or lower and still serve to secure the tibial prosthesis component 20.

The tibial prosthesis component 20 in FIG. 3 is also shown having protrusions 130a and 130b of heights 140a and 140b, respectively. As previously mentioned, the tibial prosthesis component 20 may be further secured to the tibia 30 using protrusions (e.g., protrusions 130a and 130b) that interconnect with corresponding grooves in the distal tibia 30. It should be appreciated that while protrusions 130a and 130b are shown as being cylindrical in shape, numerous other configurations would also be consistent with the principles of the invention. In addition, protrusions 130a and 130b have heights 140a and 140b above the general surface of the tibial prosthesis component 20. In one embodiment, these heights 140a and 140b are approximately 3 mm, but may also range from about 3 mm to about 10 mm. In addition, while two protrusions 130a and 130b are depicted in FIG. 3, it should equally be appreciated that fewer or more protrusions may be used consistently with the principles of the invention.

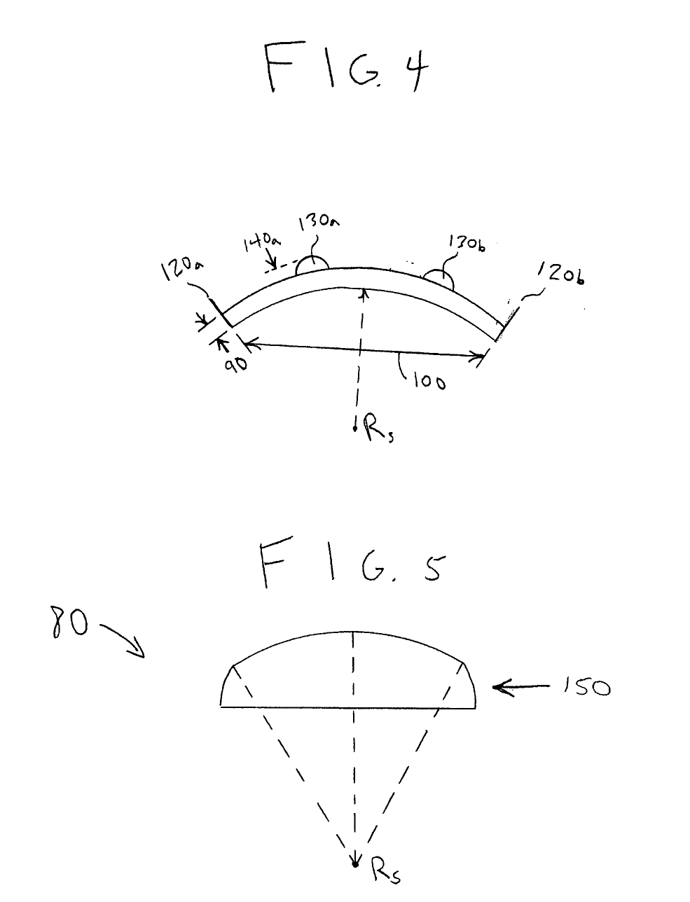

FIG. 4 is a side view of the tibial prosthesis component 20 of FIGS. 2 and 3. This angle shows how protrusions 130a and 130b are oriented and how the radius of curvature Rₛ is able to approximate the natural curvature of the distal tibia 30.

Referring now to FIG. 5, depicted is one embodiment of the bearing component 80 of FIG. 2. In this embodiment, the bearing component has the same radius of curvature Rₛ as the tibial prosthesis component 20 with which it will be in constant contact. While the bearing component 80 need not have the exact radius of curvature as a corresponding tibial component, better weight distribution, and hence less wear, may be realized as the radius of curvatures of the two components converge. In addition, as with the tibial prosthesis component 20, the bearing component 80 may also have a corresponding radius of curvature along the anterior axis indicated by arrow 150.

Although not shown in FIG. 5, the bearing component 80 may also have the previously-described grooves on its bottom flat surface, which interconnect with corresponding protrusions in the talar prosthesis component 40. This arrangement enables the tibial prosthesis component 20 to rotate or pivot in relation to the talar prosthesis component 40. FIG. 8 below will describe this embodiment is more detail.

FIG. 6 is a perspective view of one embodiment of the talar prosthesis component 40 of FIG. 2. As previously mentioned, the talar prosthesis component 40 may be secured to the talus 50 using a surgically acceptable cement such as methyl methacrylate. In addition, talar prosthesis component 40 may be further secured to the talus 50 using screws (e.g., screws 60a-60d) inserted into mounts 160a and 160b, as previously described with reference to FIG. 2. As with mounts 120a and 120b, mounts 160a and 160b may be approximately 7 mm in height, although they similarly may be larger or smaller so long as they still serve to secure the talar prosthesis component 40 to the talus 50.

The talar prosthesis component 40 may be secured to the talus 50 after a relatively small about of bone material is cut away or otherwise removed from the talus 50. While in one embodiment, approximately 3 mm of bone material may be removed, it should equally be appreciated that removal may involve more or less than 3 mm of bone material. In one embodiment, the talus 50 is cut so as to create a generally flat surface against which the correspondingly flat talar prosthesis component 40 may be secured. As with the previously described tibial prosthesis component 20, the talar prosthesis component 40 has a thickness 170, which in one embodiment is approximately 3 mm. While the surface of the prepared talus 50 has been described as being generally flat, unevenness and/or slight curvatures of the talus 50 would also be consistent with the invention, and may be compensated for by molding or otherwise matching the talar prosthesis component 40 to the talus 50 after the requisite bone material has been removed, but prior to the securing the talar prosthesis component 40 in place.

Continuing to refer to FIG. 6, the talar prosthesis component 40 is further depicted as having protrusions 180a and 180b. In one embodiment, protrusions 180a and 180b have a half-circle or half-ellipse cross section, as with previously-described protrusions 130a and 130b. It should of course be appreciated that protrusions 180a and 180b may have other cross sectional shapes. Moreover, protrusions 180a and 180b are shown as having some radius of curvature measured from a point 185 located at approximately the center of the talar prosthesis component 40. It should be appreciated that this radius of curvature may vary significantly and still be consistent with the previously-described pivoting function. In one embodiment, the point 185 represents a central pivot axis about which the talar prosthesis component 40 and a bearing component 80 rotate in relation to one another. This may be accomplished, for example, when protrusions 180a and 180b movably interlock with corresponding grooves in the bearing component, as will be described in more detail below.

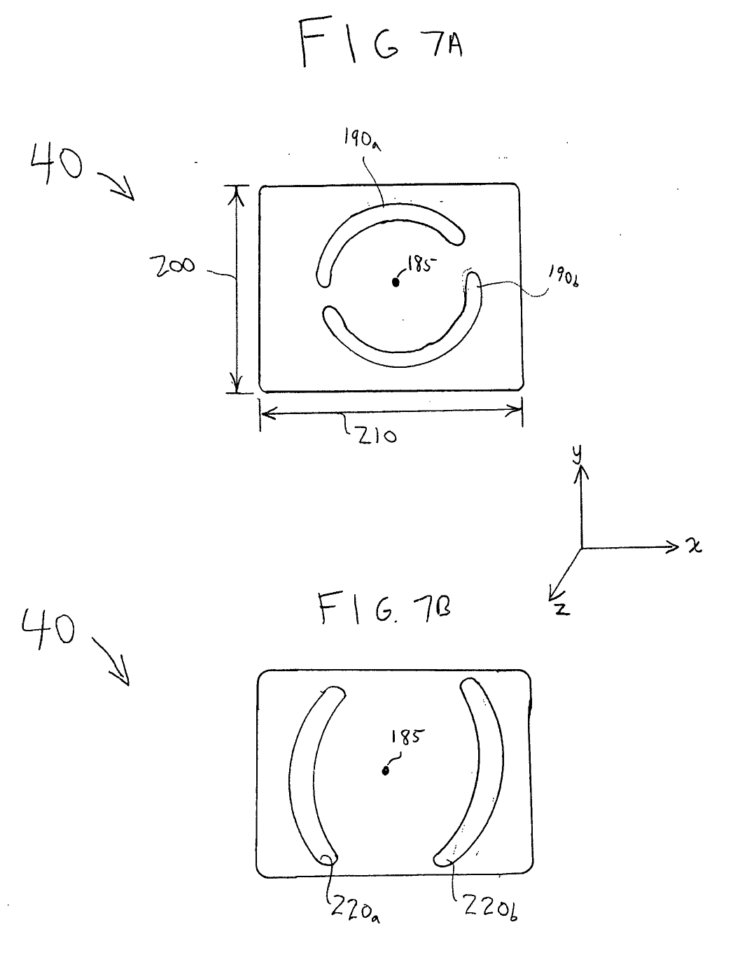

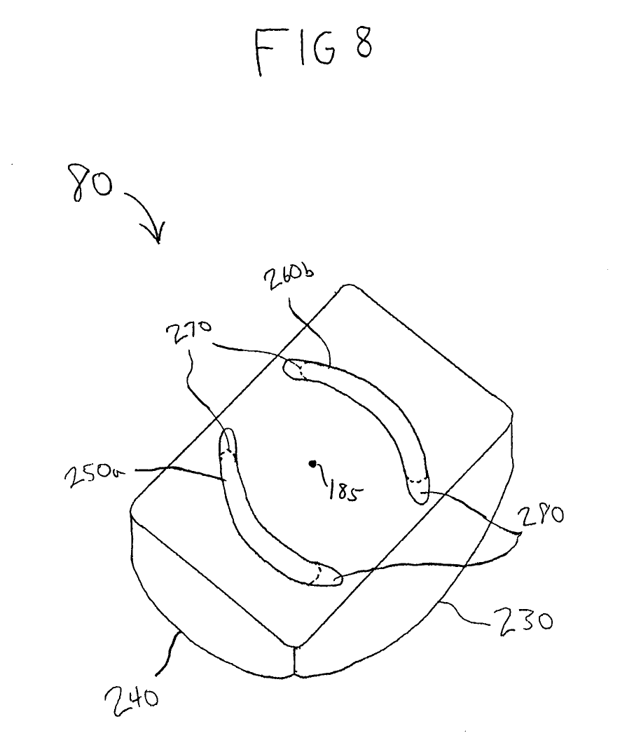

FIGS. 7A-7B depict two embodiments of the talar prosthesis component 40 viewed from the z-direction (i.e., viewed from side opposite the side to which the talus is attached). In particular, FIG. 7A is depicted as having protrusions 190a and 190b shaped as semicircles oriented about the central pivot axis point 185 of the talar prosthesis component 40. Corresponding grooves in a bearing component may be fit inside protrusions 190a and 190b to enable the ankle to pivot about the z-axis. In one embodiment, the protrusions 190a and 190b interlock with corresponding grooves in the bearing component.

The talar prosthesis component 40 of FIG. 7A has a width 200 along one side and a width 210 along the other side. In one embodiment, talar prosthesis component 40 is rectangular shaped with width 210 being greater than width 200. In one embodiment, width 200 is approximately 30 mm, while width 210 is approximately 40 mm. However, it should be appreciated that widths 200 and 210 may be larger or smaller depending on the size of the ankle to be replaced.

FIG. 7B is another embodiment of the talar prosthesis component 40 viewed from the same perspective as FIG. 7A. However, the protrusions 220a and 220b of the embodiment of FIG. 7B are not semicircles, but rather have a lower radius of curvature. Thus, it should be appreciated that the protrusions or protruding members along the top of the talar prosthesis component 40 may assume numerous sizes and configurations and still be consistent with the principles of the invention.

Referring now to FIG. 8, depicted is a perspective view of one embodiment of the bearing component 80 in which the bottom side is in view (i.e., the side that contacts the talar prosthesis component 40). As previously described with reference to FIG. 5, the bearing component 80 may have radii of curvatures along surfaces 230 and 240 so as to approximate the shape of a corresponding tibial component (e.g., tibial prosthesis component 20).

Bearing component 80 is further depicted as having curved grooves 250a and 250b. In one embodiment, grooves 250a and 250b are sized and shaped so as to accommodate corresponding protrusions from a talar component (e.g., talar prosthesis component 40). In order to provide a pivoting function, however, in one embodiment grooves 250a and 250b are longer/larger than the protrusions from a corresponding talar component that will be inserted into grooves 250a and 250b. For example, dashed lines 270 show where protrusions from a corresponding talar component may end when the bearing component 80 and talar components are interconnected. That is, spaces 280 will exist between the ends of the talar component's protrusions and the bearing component's grooves, thus enabling the protrusions and corresponding grooves to be moveably interlocked. These spaces 280 enable the bearing component (and hence a connected tibial prosthesis component) to rotate or pivot about the central axis point 185. The larger the spaces 280 are made, the greater the amount or degree of pivot is attainable. In one embodiment spaces 280 are sufficient to provide a 15 degree pivot, although more or less pivot may be desirable.

As described herein, a system, implant and kit for performing total ankle arthroplasty is provided with a tibial prosthesis component 20 being fixed to a distal tibia 30, a talar prosthesis component 40 fixed to a talus 50, and a bearing component 80 being positioned between and in contact with the tibial prosthesis component 20 and the talar prosthesis component 40.

While the preceding description has been directed to particular embodiments, it is understood that those skilled in the art may conceive modifications and/or variations to the specific embodiments described herein. Any such modifications or variations which fall within the purview of this description are intended to be included herein as well. It is understood that the description herein is intended to be illustrative only and is not intended to limit the scope of the invention.

What is claimed is:

1. A system for replacement of an ankle comprising:
a tibial prosthesis component configured to be fixed to a distal tibia end having approximately a natural radius of curvature;
a talar prosthesis component configured to be fixed to a talus;
a bearing component configured to be positioned between and in contact with the tibial prosthesis component and the talar prosthesis component.

2. The system of claim 1, wherein at least one of the tibial prosthesis component and talar prosthesis component are configured to be fixed to the distal tibia end and talus, respectively, using one or more screws.

3. The system of claim 1, wherein the tibial prosthesis component has a prosthetic radius of curvature that approximates the natural radius of curvature for the distal tibia end.

4. The system of claim 3, wherein the distal tibia end has a concave shape with said natural radius of curvature being between about 30 mm and about 40 mm.

5. The system of claim 1, wherein at least one of the tibial prosthesis component, talar prosthesis component and bearing component are configured to be inserted through one of a lateral and medial incision of the ankle.

6. The system of claim 1, wherein the tibial prosthesis component includes at least one protrusion on a major surface of the tibial prosthesis component which is configured to be in contact with the distal tibia end.

7. The system of claim 6, wherein the distal tibia end includes at least one groove into which the at least one protrusion is configured to be inserted.

8. The system of claim 6, wherein the at least one groove extends medially through the ankle.

9. The system of claim 1, wherein the talar prosthesis component has a substantially flat first major surface to which the talus is fixed.

10. The system of claim 1, wherein the talar prosthesis component includes at least one protrusion on a second major surface oriented opposite a first major surface configured to be fixed to the talus.

11. The system of claim 10, wherein the talar prosthesis component has a central pivot axis about which the talar prosthesis component and bearing component rotate in relation to each other, and the at least one protrusion is oriented about the central pivot axis.

12. The system of claim 10, wherein the bearing component includes at least one groove into which the at least one protrusion is movably interlocked.

13. The system of claim 12, wherein the at least one groove has a dimension that is larger than a corresponding dimension of the at least one protrusion such that the talar prosthesis component can rotate relative to the bearing component.

14. An implant comprising:
a tibial prosthesis component having a first major tibial surface configured to be fixed to a distal tibia end having a concave shape, and a second major tibial surface opposite the first major tibial surface;
a talar prosthesis component having a first major talar surface configured to be fixed to a talus, and a second major talar surface opposite the first major tibial surface;
a bearing component configured to be positioned between and in contact with the second major tibial surface and the second major talar surface.

15. The implant of claim 14, wherein the first major tibial surface has a prosthetic radius of curvature that approximates the concave shape of the distal tibia end.

16. The implant of claim 15, wherein the prosthetic radius of curvature is between about 30 mm and about 40 mm.

17. The implant of claim 14, wherein at least one of the tibial prosthesis component, talar prosthesis component and bearing component are configured to be inserted through one of a lateral/medial incision of the ankle.

18. The implant of claim 14, wherein the first major tibial surface includes at least one protrusion which is configured to be in contact with the distal tibia end.

19. The implant of claim 18, wherein the distal tibia end includes at least one groove into which the at least one protrusion is configured to be inserted.

20. The implant of claim 14, wherein the second major talar surface includes at least one protrusion in contact with the bearing component.

21. The implant of claim 20, wherein the talar prosthesis component has a central pivot axis about which the talar prosthesis component and bearing component rotate in relation to each other, and the at least one protrusion is oriented about the central pivot axis.

22. The implant of claim 21, wherein the bearing component includes at least one groove into which the at least one protrusion is movably interlocked.

23. The implant of claim 22, wherein the at least one groove has a dimension that is larger than a corresponding dimension of the at least one protrusion such that the talar prosthesis component can rotate relative to the bearing component.

24. A kit for performing total ankle arthroplasty comprising:
a tibial prosthesis component configured to be fixed to a distal tibia end having a concave shape;
a talar prosthesis component configured to be fixed to a talus;
a bearing component positioned configured to be between and in contact with the tibial prosthesis component and the talar prosthesis component.

25. The kit of claim 24, wherein at least one of the tibial prosthesis component and talar prosthesis component are configured to be fixed to the distal tibia end and talus, respectively, using one or more screws.

26. The kit of claim 24, wherein the tibial prosthesis component has a prosthetic radius of curvature that approximates the concave shape of the distal tibia end.

27. The kit of claim 24, wherein at least one of the tibial prosthesis component, talar prosthesis component and bearing component are configured to be inserted through one of a lateral and medial incision of the ankle.

28. The kit of claim 24, wherein the tibial prosthesis component includes at least one protrusion on a major surface of the tibial prosthesis component which is configured to be in contact with the distal tibia end.

29. The kit of claim 28, wherein the distal tibia end includes at least one groove into which the at least one protrusion is configured to be inserted.

30. The kit of claim 24, wherein the talar prosthesis component includes at least one protrusion on a second major surface oriented opposite a first major surface that is configured to be fixed to the talus.

31. The kit of claim 30, wherein the talar prosthesis component has a central pivot axis about which the talar prosthesis component and bearing component rotate in relation to each other, and the at least one protrusion is oriented about the central pivot axis.

32. The kit of claim 30, wherein the bearing component includes at least one groove into which the at least one protrusion is movably interlocked.

33. The kit of claim 32, wherein the at least one groove has a dimension that is larger than a corresponding dimension of the at least one protrusion such that the talar prosthesis component can rotate relative to the bearing component.
